(19) United States
(12) Patent Application Publication
(10) Pub. No.: US 2019/0314206 A1
(43) Pub. Date: Oct. 17, 2019

(54) Acoustic Metamaterial Aural Protection Device

(71) Applicants: Ryan Bruce Anderson, Great Mills, MD (US); Bradley Yost, Lexington Park, MD (US); Matthew Stone, Lexington Park, MD (US); Tiffany Lei, La Plata, MD (US)

(72) Inventors: Ryan Bruce Anderson, Great Mills, MD (US); Bradley Yost, Lexington Park, MD (US); Matthew Stone, Lexington Park, MD (US); Tiffany Lei, La Plata, MD (US)

(73) Assignee: United States of America as represented by the Secretary of the Navy, Patuxent River, MD (US)

(21) Appl. No.: 15/951,311

(22) Filed: Apr. 12, 2018

(51) Int. Cl. A61F 11/14; H04R 1/10

(52) U.S. Cl. CPC A61F 11/14; H04R 1/1008

(57) ABSTRACT

An acoustic metamaterial aural protection device ear cup which includes a dome having an opening, an interchangeable membrane stretched across the opening of the dome such that an airtight cavity is created, and a one-way vacuum valve; the valve is mounted to the dome, such that air can be removed from the cavity such that the membrane acts as an acoustic metamaterial, resulting in higher attenuation values at different frequencies.

### STATEMENT OF GOVERNMENT INTEREST

The invention described herein may be manufactured and used by or for the Government of the United States of America for governmental purposes without payment of any royalties thereon or therefor.

### BACKGROUND

A passive aural protection device protects a user from a high noise environment, without employing active noise reduction technology. Traditional passive over the head aural protectors incorporate a plastic ear cup or dome. Two ear cups are affixed over the user's ear and each ear cup attenuates a wide range of frequencies. These types of protectors block sound solely based on the acoustic mass law principle. To block lower frequencies, exponentially more mass is required to block the same decibel level of sound. As a result, most passive aural protectors tend to block sound more poorly at lower frequencies. Additionally, there is no well-defined way to change the frequency-specific sound attenuation values of the protectors once they have been created.

### SUMMARY

The present invention is directed to an acoustic metamaterial aural protection device ear cup which includes a dome having an opening, an interchangeable membrane stretched across the opening of the dome such that an airtight cavity is created, and a one-way vacuum valve; the valve is mounted to the dome, such that air can be removed from the cavity such that the membrane acts as an acoustic metamaterial, resulting in higher attenuation values at different frequencies.

The present invention is directed an acoustic metamaterial aural protection device which protects a user from a high noise environment, without employing active noise reduction technology.

It is a feature of the present invention to provide an acoustic metamaterial aural protection device such that the user has the ability to change the device's attenuation performance curve, or to target specific frequency ranges with increased attenuation values.

It is a feature of the present invention to provide an acoustic metamaterial aural protection device such that the device can provide superior hearing protection at specific frequencies, and can block sound at certain frequencies.

### DRAWINGS

These and other features, aspects and advantages of the present invention will become better understood with reference to the following description and appended claims, and accompanying drawings wherein:

FIG. 1A is a front view of an embodiment of the acoustic metamaterial aural protection device;

FIG. 1B is a perspective view of an ear cup of the acoustic metamaterial aural protection device of FIG. 1A;

FIG. 1C is a side cross-sectional view of an embodiment of the acoustic metamaterial aural protection device;

FIG. 1D is a side cross-sectional view of the acoustic metamaterial aural protection device of FIG. 1C with the air removed from the airtight cavity; and

FIG. 1E is front view of the membrane plate.

### DESCRIPTION

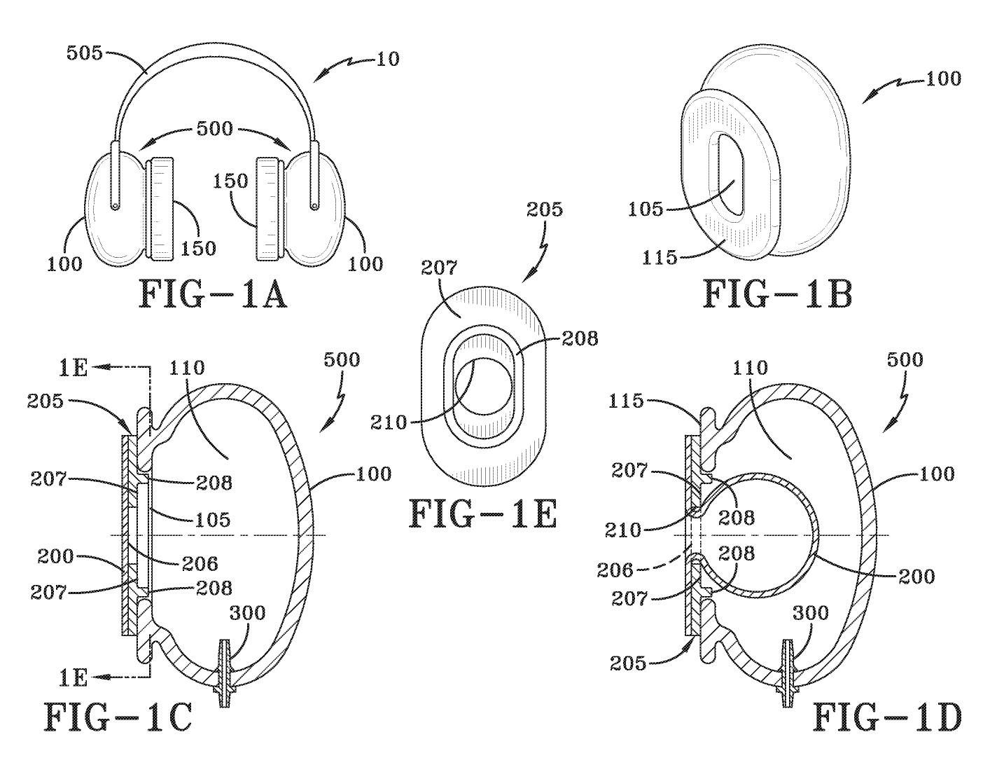

The preferred embodiments of the present invention are illustrated by way of example below and as shown in FIGS. 1A-1B. As shown in FIGS. 1A-1E, the acoustic metamaterial aural protection device 10 may include two acoustic metamaterial aural protection device ear cups 500. Each acoustic metamaterial aural protection device ear cup 500 includes a dome 100 having an opening 105, an interchangeable membrane 200, and a one-way vacuum valve 300. The interchangeable membrane is preferably latex, and is stretched across the opening 105 of the dome 100 such that an airtight cavity 110 is created, the one-way vacuum valve 300 is mounted to the dome 100, such that air can be removed from the cavity 110, such that the membrane 200 acts as an acoustic metamaterial, resulting in higher attenuation values at different frequencies.

In the description of the present invention, the invention will be discussed in a military environment; however, this invention can be utilized for any type of application that requires use of an aural protection device.

The membrane may be stretched by any means practical or by the apparatus described in the Patent Application, entitled “Tensioning Device for a Sheet of Material,” invented by the same inventors of this invention. The preferred method for attaching the membrane 200 to the dome 100, as shown in FIGS. 1C-1E, is through use of an annular membrane plate 205. The membrane plate 205 has a first surface 206 and a second surface 207. The membrane 200 (already stretched to a desired tension or unstretched) attaches to the first surface 206, while the second surface 207 attaches to the dome 100 such that the opening 105 mates with protrusion 208 and the opening 105 is covered and the membrane 200 makes the cavity 110 airtight. The membrane 200 may be attached to the first surface 206 by quick drying adhesive or any fastener that can create an airtight seal. A filler foam insert (not shown) may be placed inside the cavity 110 prior to attaching the second surface 207 to the rim 115 of the dome 100. The second surface 207 may be attached using silicone sealant or any method practicable that creates an airtight seal. A cushion 150 (preferably foam) can be added to allow a comfortable fit on a user's ear. This creates an acoustic metamaterial aural protection device ear cup 500, whereby two ear cups 500 can be attached to a headband 505, creating headgear that operates to protect hearing from various frequencies. In operation, after an adequate time for drying and/or curing, air can be removed from the cavity 110 utilizing the one-way vacuum valve 300. When air oved from the cavity HO through the one-way vacuum valve 300, the membrane 200 expands inside the dome 100 through the membrane plate opening 210, as shown in FIG. 1D.

The acoustic metamaterial aural protection device 10 can be tuned to block certain frequencies. Thus, the device is well suited for aircrew/ground crew applications, which involve loud tonal frequencies such as rotor craft and propeller aircraft platforms. These aircraft tend to produce most of their acoustic energy in a few narrow-band frequencies as opposed to broadband noise like jet noise.

When introducing elements of the present invention or the preferred embodiment(s) thereof, the articles “a,” “an,” “the,” and “said” are intended to mean there are one or more of the elements. The terms “comprising,” “including,” and “having” are intended to be inclusive and mean that there may he additional elements other than the listed elements.

Although the present invention has been described in considerable detail with reference to certain preferred embodiments thereof, other embodiments are possible. Therefore, the spirit and scope of the appended claims should not be limited to the description of the preferred embodiment(s) contained herein.

What is claimed is:

1. An acoustic metamaterial aural protection device ear cup comprising:
a dome having an opening;
an interchangeable membrane stretched across the opening of the dome such that an airtight cavity is created; and,
a one-way vacuum valve, the valve mounted to the dome, such that air can be removed from the cavity such that the membrane acts as an acoustic metamaterial, resulting in higher attenuation values at different frequencies.
